(19) United States
(12) Patent Application Publication
(10) Pub. No.: US 2026/0290495 A1
(43) Pub. Date: Sep. 24, 2026

(54) DESIGNING PROTEINS BY SIMULATED ANNEALING USING A PROTEIN STRUCTURE PREDICTION NEURAL NETWORK

(71) Applicant: GDM Holding LLC, Mountain View, CA (US)

(72) Inventors: Raphael John Lamarre Townshend, Menlo Park, CA (US); John Jumper, London (GB); David Reiman, Santa Cruz, CA (US); Simon Kohl, London (GB); Jonas Anders Adler, London (GB); Andrew W. Senior, London (GB)

(21) Appl. No.: 17/396,106

(22) Filed: Aug. 6, 2021

Related U.S. Application Data
(60) Provisional application No. 63/065,203, filed on Aug. 13, 2020.

(51) Int. Cl. G16B 15/20; G16B 15/30; G16B 40/00

(52) U.S. Cl. CPC G16B 15/20; G16B 15/30; G16B 40/00

(57) ABSTRACT

Methods, systems, and apparatus, including computer programs encoded on a computer storage medium, for designing a protein to satisfy one or more design criteria. In one aspect, a method comprises, at each of a plurality of iterations: modifying a current amino acid sequence to generate an alternative amino acid sequence; providing an input comprising a representation of the alternative amino acid sequence to a structure prediction neural network that is configured to process the input to generate an output characterizing a predicted structure of a protein specified by the alternative amino acid sequence; determining, based on the predicted structure of the protein specified by the alternative amino acid sequence, a design score characterizing how well the protein specified by the alternative amino acid sequence satisfies the design criteria; and determining, based on the design score, whether to update the current amino acid sequence to the alternative amino acid sequence.

### CROSS REFERENCE TO RELATED APPLICATION

This application claims priority under 35 U.S.C. 119 to Provisional Application No. 63/065,203, filed Aug. 13, 2020, which is incorporated by reference herein.

### BACKGROUND

This specification relates to designing proteins to satisfy one or more design criteria.

A protein is specified by one or more sequences of amino acids. An amino acid is an organic compound which includes an amino functional group and a carboxyl functional group, as well as a side-chain (i.e., group of atoms) that is specific to the amino acid. Protein folding refers to a physical process by which a sequence of amino acids folds into a three-dimensional configuration. The structure of a protein defines the three-dimensional configuration of the atoms in the amino acid sequence of the protein after the protein undergoes protein folding. When in a sequence linked by peptide bonds, the amino acids may be referred to as amino acid residues.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a protein design system implemented as computer programs on one or more computers in one or more locations that, given one or more protein design criteria, determines an amino acid sequence of a protein that is predicted to satisfy the protein design criteria.

As used throughout this specification, the term “protein” may be understood to refer to any biological molecule that is specified by one or more sequences of amino acids. For example, the term protein may be understood to refer to a protein domain (i.e., a portion of an amino acid sequence that can undergo protein folding nearly independently of the rest of the amino acid sequence) or a protein complex (i.e., that is specified by multiple associated amino acid sequences).

According to a first aspect there is provided a method performed by one or more data processing apparatus for designing a protein to satisfy one or more design criteria, the method comprising: maintaining a current amino acid sequence; and at each of a plurality of iterations: modifying the current amino acid sequence to generate an alternative amino acid sequence; providing an input comprising a representation of the alternative amino acid sequence to a structure prediction neural network that is configured to process the input to generate an output characterizing a predicted structure of a protein specified by the alternative amino acid sequence; determining, based on the predicted structure of the protein specified by the alternative amino acid sequence, a design score characterizing how well the protein specified by the alternative amino acid sequence satisfies the design criteria; and determining, based on the design score, whether to update the current amino acid sequence to the alternative amino acid sequence.

In some implementations, determining the design score characterizing how well the protein specified by the alternative amino acid sequence satisfies the design criteria comprises: determining a similarity between: (i) the predicted structure of the protein specified by the alternative amino acid sequence, and (ii) a goal protein structure; and determining the design score based at least in part on the similarity between: (i) the predicted structure of the protein specified by the alternative amino acid sequence, and (ii) the goal protein structure.

In some implementations, determining the similarity between: (i) the predicted structure of the protein specified by the alternative amino acid sequence, and (ii) the goal protein structure, comprises: determining a similarity between: (i) one or more secondary structure elements of the predicted structure specified by the alternative amino acid sequence, and (ii) one or more secondary structure elements of the goal protein structure.

In some implementations, determining the design score characterizing how well the protein specified by the alternative amino acid sequence satisfies the design criteria comprises: determining, based on the predicted structure of the protein specified by the alternative amino acid sequence, a binding affinity between: (i) the protein specified by the alternative amino acid sequence, and (ii) a binding target; and determining the design score based at least in part on the binding affinity between: (i) the protein specified by the alternative amino acid sequence, and (ii) the binding target.

In some implementations, the binding target comprises a target protein.

In some implementations, determining the design score characterizing how well the protein specified by the alternative amino acid sequence satisfies the design criteria comprises: determining a likelihood that the protein specified by the alternative amino acid sequence is associated with an adverse medical reaction; and determining the design score based at least in part on the likelihood that the protein specified by the alternative amino acid sequence is associated with the adverse medical reaction.

In some implementations, the adverse medical reaction comprises an allergic reaction.

In some implementations, determining the design score characterizing how well the protein specified by the alternative amino acid sequence satisfies the design criteria comprises: determining a predicted structural stability score characterizing a predicted structural stability of the protein specified by the alternative amino acid sequence; and determining the design score based at least in part on the predicted structural stability score.

In some implementations, modifying the current amino acid sequence to generate an alternative amino acid sequence comprises, for one or more positions in the current amino acid sequence: randomly modifying an identity of an amino acid at the position in the current amino acid sequence.

In some implementations, modifying the current amino acid sequence to generate the alternative amino acid sequence comprises: adding one or more additional positions to the current amino acid sequence, wherein each additional position is occupied by a respective amino acid.

In some implementations, modifying the current amino acid sequence to generate the alternative amino acid sequence comprises: removing one or more positions from the current amino acid sequence.

In some implementations, a length of the alternative amino acid sequence is different than a length of the current amino acid sequence.

In some implementations, the structure prediction neural network is a feed-forward neural network.

In some implementations, the structure prediction neural network processes the input comprising the representation of the alternative amino acid sequence to generate the output characterizing the predicted structure of the protein specified by the alternative amino acid sequence in less than five seconds.

In some implementations, the structure prediction neural network processes an input comprising a respective embedding of an amino acid in each position of the alternative amino acid sequence.

In some implementations, determining whether to update the current amino acid sequence to the alternative amino acid sequence comprises: obtaining a design score characterizing how well a protein specified by the current amino acid sequence satisfies the design criteria; and determining whether to update the current amino acid sequence to the alternative amino acid sequence based on: (i) the design score for the alternative amino acid sequence, and (ii) the design score for the current amino acid sequence.

In some implementations, determining whether to update the current amino acid sequence to the alternative amino acid sequence based on: (i) the design score for the alternative amino acid sequence, and (ii) the design score for the current amino acid sequence, comprises: determining an acceptance probability based on: (i) the design score for the alternative amino acid sequence, and (ii) the design score for the current amino acid sequence; and updating the current amino acid sequence to the alternative amino acid sequence with probability defined by the acceptance probability.

In some implementations, the method further comprises, after a final iteration of the one or more iterations, providing an output comprising data defining the current amino acid sequence.

In another aspect, there is provided a system comprising: one or more computers; and one or more storage devices communicatively coupled to the one or more computers, wherein the one or more storage devices store instructions that, when executed by the one or more computers, cause the one or more computers to perform the operations of the methods described herein.

In another aspect, there are provided one or more non-transitory computer storage media storing instructions that when executed by one or more computers cause the one or more computers to perform the operations of the methods described herein.

Particular embodiments of the subject matter described in this specification can be implemented so as to realize one or more of the following advantages.

The protein design system described in this specification can iteratively optimize an amino acid sequence to encourage the protein specified by the amino acid sequence to satisfy one or more design criteria. The protein design system can optimize the amino acid sequence to achieve any of a variety of possible design criteria, some or all of which may be based on the structure of the protein specified by the amino acid sequence. For example, the protein design system can optimize the amino acid sequence to encourage the corresponding protein to have a specified protein structure, i.e., after protein folding. As another example, the protein design system can optimize the amino acid sequence to encourage the corresponding protein to have a desired level of structural stability, where the structural stability characterizes the likelihood that the protein will maintain its folded structure rather than reverting to its denatured (unfolded) state. As another example, the protein design system can optimize the amino acid sequence to encourage the corresponding protein to have a low likelihood of being associated with an adverse medical reaction, e.g., an allergic reaction, if a drug based on the protein is administered to a patient. As another example, the protein design system can optimize the amino acid sequence to encourage the corresponding protein to have a desired strength of binding interaction with a binding target, e.g., another protein or a lipid.

The protein design system can optimize an amino acid sequence to encourage the corresponding protein to satisfy “high-level” design criteria that can be easily specified by a user of the system, e.g., design criteria requiring that the structure of the protein include a specified number of secondary structure elements. Examples of secondary structure elements include alpha helices and beta sheets. In contrast, some other systems may require that a user specify a target protein structure that should be achieved by the protein, e.g., by specifying the coordinates and orientations of individual atoms or amino acids in the protein, which may be more difficult and time-consuming.

Given a set of design criteria, the protein design system can rapidly generate one or more amino acid sequences that each specify a respective protein that is predicted to satisfy the design criteria. The protein design system can achieve this rapidity by using a protein structure prediction neural network that can predict the structure of a protein, e.g., in a matter of seconds by one forward pass through the protein structure prediction neural network. By enabling rapid protein design, the protein design system can facilitate and accelerate areas of biochemical research and engineering that involve proteins, e.g., drug development.

The protein design system allows the length of the amino acid sequence being optimized to vary during the optimization, i.e., to enable the corresponding protein to better satisfy the design criteria. In contrast, some conventional systems may be configured to receive the length of the amino acid sequence as an input to the system, and thereafter generate an output amino acid sequence having the length specified by the input. Automatically optimizing the length of the amino acid sequence may enable the protein design system described in this specification to design proteins more effectively than some conventional systems, e.g., that require the length of the amino acid sequence to be specified in advance.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example protein design system.

FIG. 2 is an illustration of an unfolded protein and a folded protein.

FIG. 3 is a flow diagram of an example process for designing a protein to satisfy one or more design criteria.

Like reference numbers and designations in the various drawings indicate like elements.

### DETAILED DESCRIPTION

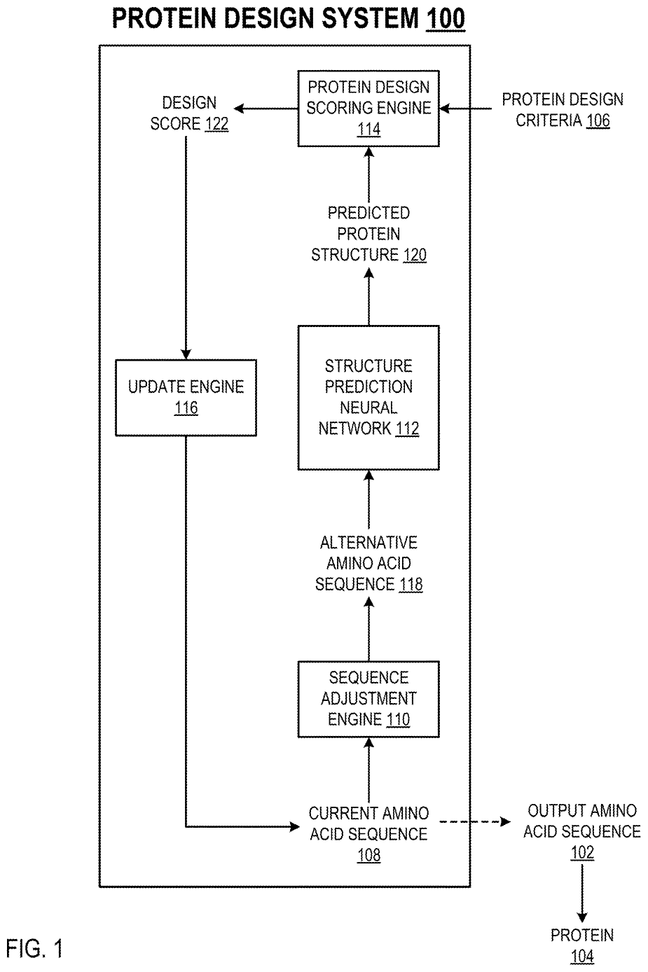

FIG. 1 shows an example protein design system 100. The protein design system 100 is an example of a system implemented as computer programs on one or more computers in one or more locations in which the systems, components, and techniques described below are implemented.

The system 100 is configured to determine an “output” amino acid sequence 102 that specifies a protein 104 which is predicted to satisfy one or more protein design criteria 106. The design criteria 106 specify one or more desired attributes of the protein 104, e.g., the design criteria 106 may specify a target structure of the protein 104. The design criteria 106 may be provided to the system 100 by a user of the system 100, e.g., through an interface made available by the system 100. The user may be a remote user that provides the design criteria to the system 100 over a communications network, e.g., the internet.

To determine the output amino acid sequence 102, the system 100 maintains and iteratively adjusts a “current” amino acid sequence 108 over a sequence of optimization iterations to encourage the protein specified by the current amino acid sequence 108 to satisfy the design criteria 106. Prior to the first optimization iteration, the system 100 may initialize the current amino acid sequence 108, e.g., by randomly sampling the identity of the amino acid in each position of the current amino acid sequence 108 from a set of possible amino acids. The set of possible amino acids may include, e.g., alanine, arginine, asparagine, etc. The system 100 may randomly sample the initial length of the current amino acid sequence 108 from a range of possible lengths, e.g., the integer values in the range [50, 1000]. The “length” of an amino acid sequence may refer to the number of positions in the amino acid sequence. Rather than the current amino acid sequence 108 being randomly initialized prior to the first optimization iteration, the current amino acid sequence 108 may be initialized to the amino acid sequence of a known protein, e.g., that is specified by a user of the system 100.

The system 100 uses a sequence adjustment engine 110, a structure prediction neural network 112, a protein design scoring engine 114, and an update engine 116, which will each be described in more detail next, to iteratively adjust the current amino acid sequence 108 over multiple optimization iterations.

At each optimization iteration, the sequence adjustment engine 110 processes the current amino acid sequence 108 to generate an alternative amino acid sequence 118, i.e., that is different than the current amino acid sequence 108. The sequence adjustment engine 110 can generate the alternative amino acid sequence 118 by modifying the current amino acid sequence 108 in any of a variety of ways. A few example techniques for modifying the current amino acid sequence 108 to generate the alternative amino acid sequence 118 are described in more detail next.

In one example, the sequence adjustment engine 110 may generate the alternative amino acid sequence 118 by randomly selecting a predetermined number of positions in the current amino acid sequence 108, and randomly modifying the respective identity of the amino acid at each selected position. The sequence adjustment engine 110 may randomly modify the identity of an amino acid at a position in the current amino acid sequence 108, e.g., by randomly sampling a new identity of the amino acid in accordance with a probability distribution over the set of possible amino acids. The probability distribution over the set of possible amino acids may be, e.g., a uniform probability distribution that assigns an equal likelihood to each possible amino acid. In another example, the probability distribution over the set of possible amino acids may assign a respective likelihood to each possible amino acid based on the frequency of occurrence of the possible amino acid in the amino acid sequences of proteins in a set of known proteins. In particular, the probability distribution may assign a higher likelihood to possible amino acids that occur more frequency in the amino acid sequences of proteins in the set of known proteins.

In another example, the sequence adjustment engine 110 may generate the alternative amino acid sequence 118 by increasing or decreasing the length of the current amino acid sequence 108.

To increase the length of the current amino acid sequence 108, the sequence adjustment engine 110 may add one or more new positions to the current amino acid sequence 108, e.g., at randomly selected locations within the current amino acid sequence 108. The sequence adjustment engine 110 may determine the number of new positions to be added to the current amino acid sequence 108, e.g., by randomly sampling the number of new positions to be added from a predetermined set of non-negative integer values. The sequence adjustment engine 110 may determine the respective identity of the amino acid at each new position added to the current amino acid sequence 108, e.g., by randomly sampling the identity of the amino acid in accordance with a probability distribution over a set of possible amino acids. Example techniques for randomly sampling from a set of possible amino acids are described in more detail above.

To decrease the length of the current amino acid sequence 108, the sequence adjustment engine 110 may remove one or more positions from the current amino acid sequence 108. For example, the sequence adjustment engine 110 may randomly sample the number of positions to be removed from a predetermined set of non-negative integer values, and randomly select the positions to be removed from within the current amino acid sequence 108.

Generally, the sequence adjustment engine 110 may generate the alternative amino acid sequence 118 by modifying the current amino acid sequence 108 in a combination of ways. For example, the sequence adjustment engine 110 may modify the current amino acid sequence by a combination of adding one or more new positions, removing one or more existing positions, and modifying the identities of amino acids at one or more existing positions in the current amino acid sequence.

At each optimization iteration, the structure prediction neural network 112 processes a representation of the alternative amino acid sequence 118 to generate a set of structure parameters that define the predicted structure 120 of the protein specified by the alternative amino acid sequence 118 (i.e., the “alternative protein”). The predicted structure 120 defines an estimate of a three-dimensional (3-D) configuration of the atoms in the alternative protein after the alternative protein undergoes protein folding.

The output of the structure prediction neural network 112 may define the predicted structure 120 of the alternative protein, e.g., by a set of structure parameters. In some implementations, the structure parameters are a sequence of three-dimensional (3D) numerical coordinates (e.g., represented as 3D vectors) where each coordinate represents the position (in some given frame of reference) of a corresponding atom in an amino acid of the alternative protein. For example, the structure parameters may be a sequence of 3D numerical coordinates representing the respective positions of the alpha carbon atoms in the amino acids of the alternative protein. An alpha carbon atom, which is referred to in this specification as a backbone atom, refers to a carbon atom in an amino acid to which the amino functional group, the carboxyl functional group, and the side-chain are bonded. In some implementations, the structure parameters are a sequence of torsion (i.e., dihedral) angles between specific atoms in the amino acids of the alternative protein. For example, the structure parameters may be a sequence of phi (φ), psi (ψ), and omega (ω) dihedral angles between the backbone atoms in amino acids of the alternative protein.

To generate the predicted protein structure 120 corresponding to the alternative amino acid sequence 118, the structure prediction neural network 112 may process an input that includes a representation of the alternative amino acid sequence 118. The representation of the alternative amino acid sequence may include a respective embedding (i.e., an ordered collection of numerical values, e.g., a vector or matrix of numerical values) corresponding to each position in the alternative amino acid sequence. The respective embedding corresponding to each position in the alternative amino acid sequence 118 may be, e.g., a one-hot vector that defines the identity of the amino acid in the position in the alternative amino acid sequence 118. A one-hot vector has a different component corresponding to each possible amino acid (e.g., of a predetermined number of possible amino acids). A one-hot vector representing a particular amino acid has value one (or some other predetermined value) in the component corresponding to the particular amino acid and value zero (or some other predetermined value) in the other components.

The structure prediction neural network 112 may have any appropriate neural network architecture that enables it to perform its described function. For example, the structure prediction neural network 112 may be a feed-forward neural network that can process an input characterizing an amino acid sequence to generate a predicted structure of the corresponding protein in seconds, e.g., five seconds or less. The structure prediction neural network may have an architecture that includes any appropriate types of neural network layers arranged in any appropriate configuration (e.g., as a linear sequence of layers). For example, the structure prediction neural network may include fully-connected neural network layers, convolutional neural network layers, pooling neural network layers, self-attention neural network layers, or a combination thereof. The structure prediction neural network 112 may be trained using machine learning techniques, e.g., stochastic gradient descent, on a set of training data that includes multiple training examples. Each training example may specify: (i) a training input that includes a representation of an amino acid sequence, and (ii) a target output that defines the protein structure corresponding to the amino acid sequence. The target output may specify the output that should be generated by processing the corresponding training input and may be determined, e.g., using physical experiments, e.g., x-ray crystallography.

At each optimization iteration, the protein design scoring engine 114 determines a design score 122 that characterizes how well the alternative protein specified by the alternative amino acid sequence 118 satisfies the design criteria 106 based at least in part on the predicted structure 120 of the alternative protein. Generally, the design criteria 106 specify one or more desired attributes of the protein 104 specified by the output amino acid sequence 102. The scoring engine 114 may determine a respective score for each attribute specified by the design criteria 106 that characterizes whether (or the degree to which) the alternative protein possesses the attribute. The scoring engine 114 may determine the design score 122 as a function, e.g., a weighted linear combination, of the attribute scores. A few examples of attribute scores, including structure scores, binding scores, adverse reaction scores, and stability scores, are described in more detail next.

In one example, one of the attributes specified by the design criteria 106 may be that the protein 104 specified by the output amino acid sequence 102 should have a “goal” (i.e., target) protein structure. The scoring engine 114 may determine the score for this attribute (a structure score) that characterizes a similarity between: (i) the predicted structure 120 of the alternative protein, and (ii) the goal protein structure.

In some implementations, the goal protein structure may be specified by a set of goal structure parameters. The goal structure parameters may include, e.g., a sequence of dihedral angles between the backbone atoms in amino acids in the goal protein structure, or respective coordinates defining the 3-D spatial positions of the backbone atoms in the goal protein structure.

In some implementations, the goal protein structure may be specified in terms of secondary protein structure elements. A secondary protein structure element defines a structure that may be assumed by a sub-sequence of the amino acid sequence of a protein, i.e., by a local segment of the protein. Examples of secondary protein structure elements include alpha helices, beta sheets, beta turns, and omega loops. The goal protein structure may be expressed in terms of secondary protein structure elements, e.g., by indicating a respective number of each of one or more secondary protein structure elements that should occur in the goal protein structure, or by indicating a sequence of respective secondary protein structure elements that should occur in the goal protein structure. In particular examples, the goal protein structure may be required to include a first number of alpha helices and a second number of beta sheets, or the goal protein structure may be required to include an alpha helix followed by a beta sheet.

In another example, one of the attributes specified by the design criteria 106 may be that the protein 104 specified by the output amino acid sequence 102 should bind selectively and specifically to a “binding target” (or “ligand”). The binding target may be, e.g., an ion (e.g., aluminum—Al 3+), a small molecule (e.g., adenosine triphosphate—ATP), or a macromolecule (e.g., another protein or a lipid). In this example, the scoring engine 114 may determine the score for this attribute (a binding (affinity) score) that characterizes the predicted strength of the binding interaction between: (i) the alternative protein specified by the alternative amino acid sequence 118, and (ii) the binding target.

In another example, one of the attributes specified by the design criteria 106 may be that the protein 104 specified by the output amino acid sequence 102 should have a low likelihood of being associated with an adverse medical reaction, e.g., an allergic reaction, nerve injury, organ injury, or impaired vision or hearing. In this example, the scoring engine 114 may determine the score for this attribute (an adverse reaction score) based at least in part on a predicted likelihood that the alternative protein having the predicted structure 120 is associated with an adverse medical reaction.

In another example, one of the attributes specified by the design criteria 106 may be that the protein 104 specified by the output amino acid sequence 102 should have a desired structural stability. The structural stability of a protein may refer to the likelihood that the protein will maintain its folded structure rather than reverting to its denatured (i.e., unfolded) state. In some cases, the structural stability of a protein may refer more specifically to, e.g., the thermal stability of the protein (i.e., the likelihood that the protein will maintain its folded structure at elevated temperatures), or proteolytic stability of the protein (i.e., the likelihood that the protein will maintain its folded structure in the presence of proteolytic enzymes that catalyze the breakdown of proteins). In this example, the scoring engine 114 may determine the score for this attribute (a stability score) based at least in part on a predicted structural stability of the protein.

The scoring engine 114 may determine the attribute scores in any of a variety of ways. A few example techniques for determining the attribute scores are described in more detail next.

The scoring engine 114 may determine the structure score based on a similarity measure between: (i) the predicted structure 120 of the alternative protein, and (ii) the goal protein structure. In one example, the similarity measure may be a root-mean-square-deviation (RMSD) between the structure parameters defining the predicted structure 120 and structure parameters defining the goal protein structure. In another example, the similarity measure may be based on how many of the secondary protein structure elements of the goal protein structure are achieved in the predicted structure of the alternative protein.

The scoring engine 114 may determine certain attribute scores, e.g., the binding scores, adverse reaction scores, and stability scores, by processing the predicted protein structure 120 and/or a representation of the alternative amino acid sequence 118 using an attribute prediction machine learning model that is trained to generate the attribute score. The attribute prediction machine learning model may be trained on a set of training examples that each specify: (i) a respective training input that includes data characterizing a protein structure and/or an amino acid sequence, and (ii) a target attribute score that should be generated by the attribute prediction machine learning model by processing the training input. The target attribute score may be, e.g., a binding score, an adverse reaction score, or a stability score. The target attribute scores may be determined, e.g., by physical experiments.

The attribute prediction machine learning model may be trained using an appropriate training technique to minimize an error between: (i) predicted attribute scores generated by the attribute prediction neural network, and (ii) target attribute scores specified by the training examples. The attribute prediction machine learning model may be, e.g., a neural network, a support vector machine, a random forest, a regression model, or any other appropriate machine learning model. An attribute prediction neural network may have any appropriate neural network architecture that enables it to perform its described function, e.g., an architecture that includes one or more fully-connected neural network layers, convolutional neural network layers, pooling neural network layers, self-attention neural network layers, or a combination thereof. An attribute prediction neural network may be trained using appropriate neural network training techniques, e.g., stochastic gradient descent.

In a particular example, the scoring engine 114 may use an attribute prediction neural network to generate the binding score between: (i) the alternative protein, and (ii) a binding target that includes a target protein, using a binding prediction neural network. The binding prediction neural network may be configured to process respective representations of: (i) the predicted structure 120 of the alternative protein, and (ii) the (predicted) structure of the target protein, to generate the binding score for the alternative protein and the target protein.

At each optimization iteration, the update engine 116 determines whether to update the current amino acid sequence 108 to the alternative amino acid sequence 118 based on the respective design scores 122 for: (i) the alternative amino acid sequence 118, and (ii) the current amino acid sequence 108. The system 100 may have previously determined the design score for the current amino acid sequence 108, i.e., at the iteration where the current amino acid sequence 108 was generated, and thereafter maintained the design score for the current amino acid sequence. Therefore, rather than regenerating the design score for the current amino acid sequence 108 at the current iteration, the system 100 may retrieve and use the previously generated design score for the current amino acid sequence 108.

The update engine 116 may determine whether to update the current amino acid sequence 108 to the alternative amino acid sequence 118 using a simulated annealing optimization technique. More specifically, the update engine 116 may determine an “acceptance probability”, e.g., represented as a numerical value in the range [0,1], that defines a likelihood for updating the current amino acid sequence 108 to the alternative amino acid sequence 118. The update engine 116 then updates the current amino acid sequence 108 to the alternative amino acid sequence 118 with probability defined by the acceptance probability.

The update engine 116 may determine the acceptance probability based on: (i) a “temperature” parameter (which may be represented as a numerical value), (ii) the design score for the current amino acid sequence 108, and (iii) the design score for the alternative amino acid sequence 118. Generally, higher values of the temperature parameter may cause the system 100 to implement a more “exploratory” optimization policy, while lower values of the temperature parameter may cause the system 100 to implement a more “greedy” optimization policy. For example, for a high value of the temperature parameter, the update engine 116 may determine a high acceptance probability even if the design score for the alternative amino acid sequence 118 is inferior to (e.g., lower than) the design score for the current amino acid sequence 108. In one example, the update engine 116 may determine the acceptance probability a as:

$$a = \{ \begin{matrix}1 & if {d}^{′} > d \\ exp ( \frac{{d}^{′} - d}{T + ϵ} ) & otherwise\end{matrix} \qquad ( 4 )$$

where d′ denotes the design score for the alternative amino acid sequence 118, d denotes the design score for the current amino acid sequence 108, where a higher design score for an amino acid sequence indicates that the protein specified by the amino acid sequence better satisfies the design criteria, T denotes the temperature parameter, and E denotes a small positive constant to improve numerical stability. Other techniques for determining the acceptance probability are possible, and the technique defined by equation (4) is provided for illustrative purposes only.

The system 100 may determine that the current optimization iteration is the final optimization iteration once a termination criterion is satisfied, e.g., once a predetermined number of optimization iterations have been performed, or once the design score for the current amino acid sequence achieves (e.g., exceeds) a predefined threshold.

After the final optimization iteration, the system 100 selects an amino acid sequence generated during the optimization, i.e., at one of the optimization iterations, as the output amino acid sequence 102 specifying the protein 104. For example, the system 100 may select the current amino acid sequence at the final optimization iteration as the output amino acid sequence 102. As another example, the system 100 may select the amino acid sequence having the best (e.g., highest) design score among all the amino acid sequences generated during the optimization as the output amino acid sequence 102.

In some cases, rather than selecting a single output amino acid sequence 102, the system 100 may select multiple output amino acid sequences 102 specifying respective proteins 104. For example, the system 100 may select a predefined number of amino acid sequences having the best (e.g., highest) design scores from among all the amino acid sequences generated during the optimization as respective output amino acid sequences 102.

Optionally, the system 100 may repeatedly perform the optimization procedure to generate multiple output amino acid sequences, each of which may be different, e.g., due to randomness in the way the amino acid sequence is initialized at the first iteration, or due to randomness during the optimization procedure. The randomness during the optimization procedure may arise, e.g., by probabilistically determining whether the current amino acid sequence 108 should be updated at each iteration, as described above.

Generally, the protein 104 specified by the output amino acid sequence 102 generated by the system 100 may not strictly satisfy some, or any, of the protein design criteria 106 that defined the optimization performed by the system 100. For example, if the protein design criteria 106 specify target structure parameters for the structure of the protein 104, the predicted structure parameters defining the predicted structure of the protein 104 may not precisely match the target structure parameters. The protein 104 may not strictly satisfy all the protein design criteria 106, e.g., because biochemical constraints may prevent the protein from strictly satisfying the design criteria 106, or because the optimization performed by the system 100 may have converged to an undesirable local optimum of the design scores.

A protein having the output amino acid sequence 102 generated by the system 100 may be experimentally synthesized and validated, e.g., to determine by physical experiments whether the synthesized protein actually achieves the design criteria 106 provided to the system 100. Proteins designed by the system 100 can be used for any of a variety of practical applications, e.g., as drugs to be administered to patients to achieve therapeutic effects, or as enzymes to catalyze industrial processes.

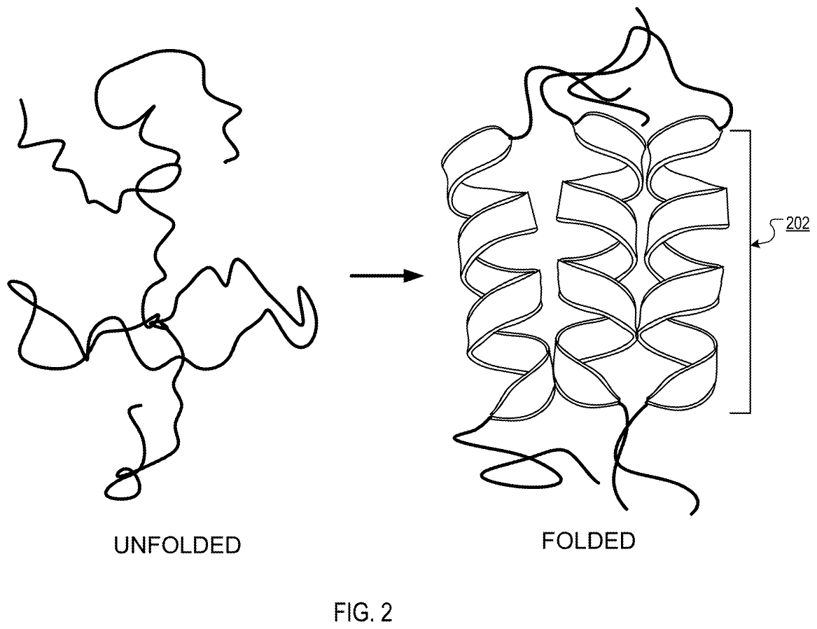

FIG. 2 is an illustration of an unfolded protein and a folded protein. The unfolded protein is a random coil of amino acids. The unfolded protein undergoes protein folding and folds into a 3D configuration. Protein structures often include stable local folding patterns such alpha helices (e.g., as depicted by 202) and beta sheets.

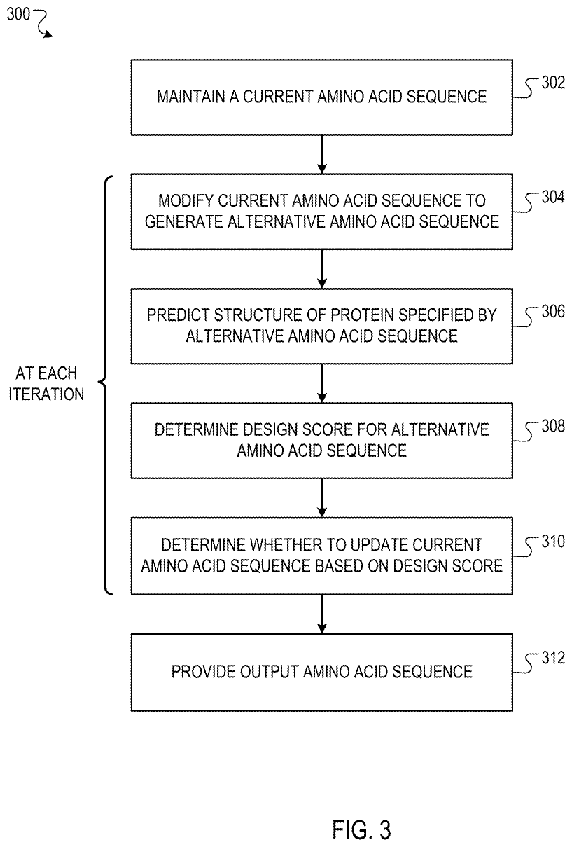

FIG. 3 is a flow diagram of an example process 300 for designing a protein to satisfy one or more design criteria. For convenience, the process 300 will be described as being performed by a system of one or more computers located in one or more locations. For example, a protein design system, e.g., the protein design system 100 of FIG. X, appropriately programmed in accordance with this specification, can perform the process 300.

The system maintains a current amino acid sequence (302).

The system performs steps 304-310, which are described in more detail next, at each of multiple iterations.

The system modifies the current amino acid sequence to generate an alternative amino acid sequence (304).

The system provides an input that includes a representation of the alternative amino acid sequence to a structure prediction neural network that is configured to process the input to generate an output characterizing a predicted structure of a protein specified by the alternative amino acid sequence (306).

The system determines, based on the predicted structure of the protein specified by the alternative amino acid sequence, a design score characterizing how well the protein specified by the alternative amino acid sequence satisfies the design criteria (308).

The system determines, based on the design score, whether to update the current amino acid sequence to the alternative amino acid sequence (310).

After a final iteration of the multiple iterations, the system provides an output amino acid sequence, e.g., the current amino acid sequence at the final iteration (312).

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
computationally designing an amino acid sequence of a binder protein of a drug that targets a binding target that is involved in a disease process, wherein computationally designing the amino acid sequence of the binder protein comprises performing, by one or more computers, operations comprising:
maintaining a current amino acid sequence of the binder protein; and
at each of a plurality of iterations:
modifying the current amino acid sequence of the binder protein to generate an alternative amino acid sequence of the binder protein;
providing an input comprising a representation of the alternative amino acid sequence of the binder protein to a structure prediction neural network that is configured to process the input to generate an output characterizing a predicted structure of the binder protein specified by the alternative amino acid sequence;
determining, based on the predicted structure of the binder protein specified by the alternative amino acid sequence, a design score for the alternative amino acid sequence characterizing at least a predicted binding affinity of the binder protein specified by the alternative amino acid sequence for the binding target; and
determining, based on the design score, whether to update the current amino acid sequence of the binder protein to the alternative amino acid sequence, comprising:
obtaining a design score for the current amino acid sequence characterizing at least a predicted binding affinity of the binder protein specified by the current amino acid sequence for the binding target;
determining an acceptance probability based on: (i) the design score for the alternative amino acid sequence, (ii) the design score for the current amino acid sequence, and (iii) a temperature parameter, wherein higher values of the temperature parameter determine a higher acceptance probability even if the design score for the alternative amino acid sequence is inferior to the design score for the current amino acid sequence; and
updating the current amino acid sequence of the binder protein to the alternative amino acid sequence with probability defined by the acceptance probability; and
selecting, using the one or more computers, an output amino acid sequence for the binder protein of the drug that targets the binding target based on the design scores generated over the plurality of iterations, wherein the output amino acid sequence has a superior design score as compared to a plurality of other amino acid sequences generated over the plurality of iterations; and
providing the selected output amino acid sequence for the binder protein for synthesis.

2. The method of claim 1, wherein determining the design score comprises:
determining a similarity between: (i) the predicted structure of the protein specified by the alternative amino acid sequence, and (ii) a goal protein structure; and
determining the design score based at least in part on the similarity between: (i) the predicted structure of the protein specified by the alternative amino acid sequence, and (ii) the goal protein structure.

3. The method of claim 2, wherein determining the similarity between: (i) the predicted structure of the protein specified by the alternative amino acid sequence, and (ii) the goal protein structure, comprises:
determining a similarity between: (i) one or more secondary structure elements of the predicted structure specified by the alternative amino acid sequence, and (ii) one or more secondary structure elements of the goal protein structure.

4. (canceled)

5. The method of claim 1, wherein the binding target comprises a target protein.

6. The method of claim 1, wherein determining the design score comprises:
determining a likelihood that the protein specified by the alternative amino acid sequence is associated with an adverse medical reaction; and
determining the design score based at least in part on the likelihood that the protein specified by the alternative amino acid sequence is associated with the adverse medical reaction.

7. The method of claim 6, wherein the adverse medical reaction comprises an allergic reaction.

8. The method of claim 1, wherein determining the design score comprises:
determining a predicted structural stability score characterizing a predicted structural stability of the protein specified by the alternative amino acid sequence; and
determining the design score based at least in part on the predicted structural stability score.

9. The method of claim 1, wherein modifying the current amino acid sequence to generate an alternative amino acid sequence comprises, for one or more positions in the current amino acid sequence:
randomly modifying an identity of an amino acid at the position in the current amino acid sequence.

10. The method of claim 1, wherein modifying the current amino acid sequence to generate the alternative amino acid sequence comprises:
adding one or more additional positions to the current amino acid sequence, wherein each additional position is occupied by a respective amino acid.

11. The method of claim 1, wherein modifying the current amino acid sequence to generate the alternative amino acid sequence comprises:
removing one or more positions from the current amino acid sequence.

12. The method of claim 10, wherein a length of the alternative amino acid sequence is different than a length of the current amino acid sequence.

13. The method of claim 1, wherein the structure prediction neural network is a feed-forward neural network.

14. The method of claim 1, wherein the structure prediction neural network processes the input comprising the representation of the alternative amino acid sequence to generate the output characterizing the predicted structure of the protein specified by the alternative amino acid sequence in less than five seconds.

15. The method of claim 1, wherein the structure prediction neural network processes an input comprising a respective embedding of an amino acid in each position of the alternative amino acid sequence.

16. (canceled)

17. (canceled)

18. The method of claim 1, further comprising, after a final iteration of the one or more iterations, providing an output comprising data defining the current amino acid sequence.

19. A system comprising:
one or more computers; and
one or more storage devices communicatively coupled to the one or more computers, wherein the one or more storage devices store instructions that, when executed by the one or more computers, cause the one or more computers to perform operations for computational drug or therapeutic design, the operations comprising:
computationally designing an amino acid sequence of a binder protein of a drug that targets a binding target that is involved in a disease process, wherein computationally designing the amino acid sequence of the binder protein comprises performing, by one or more computers, operations comprising:
maintaining a current amino acid sequence of the binder protein; and
at each of a plurality of iterations:
modifying the current amino acid sequence of the binder protein to generate an alternative amino acid sequence of the binder protein;
providing an input comprising a representation of the alternative amino acid sequence of the binder protein to a structure prediction neural network that is configured to process the input to generate an output characterizing a predicted structure of the binder protein specified by the alternative amino acid sequence;
determining, based on the predicted structure of the binder protein specified by the alternative amino acid sequence, a design score for the alternative amino acid sequence characterizing at least a predicted binding affinity of the binder protein specified by the alternative amino acid sequence for the binding target; and
determining, based on the design score, whether to update the current amino acid sequence of the binder protein to the alternative amino acid sequence, comprising:
obtaining a design score for the current amino acid sequence characterizing at least a predicted binding affinity of the binder protein specified by the current amino acid sequence for the binding target;
determining an acceptance probability based on: (i) the design score for the alternative amino acid sequence, (ii) the design score for the current amino acid sequence, and (iii) a temperature parameter, wherein higher values of the temperature parameter determine a higher acceptance probability even if the design score for the alternative amino acid sequence is inferior to the design score for the current amino acid sequence; and
updating the current amino acid sequence of the binder protein to the alternative amino acid sequence with probability defined by the acceptance probability; and
selecting, using the one or more computers, an output amino acid sequence for the binder protein of the drug that targets the binding target based on the design scores generated over the plurality of iterations, wherein the output amino acid sequence has a superior design score as compared to a plurality of other amino acid sequences generated over the plurality of iterations; and
providing the selected output amino acid sequence for the binder protein for synthesis.

20. One or more non-transitory computer storage media storing instructions that when executed by one or more computers cause the one or more computers to perform operations for computational drug or therapeutic design, the operations comprising:
computationally designing an amino acid sequence of a binder protein of a drug that targets a binding target that is involved in a disease process, wherein computationally designing the amino acid sequence of the binder protein comprises performing, by one or more computers, operations comprising:
maintaining a current amino acid sequence of the binder protein; and
at each of a plurality of iterations:
modifying the current amino acid sequence of the binder protein to generate an alternative amino acid sequence of the binder protein;
providing an input comprising a representation of the alternative amino acid sequence of the binder protein to a structure prediction neural network that is configured to process the input to generate an output characterizing a predicted structure of the binder protein specified by the alternative amino acid sequence;
determining, based on the predicted structure of the binder protein specified by the alternative amino acid sequence, a design score for the alternative amino acid sequence characterizing at least a predicted binding affinity of the binder protein specified by the alternative amino acid sequence for the binding target; and
determining, based on the design score, whether to update the current amino acid sequence of the binder protein to the alternative amino acid sequence, comprising:
obtaining a design score for the current amino acid sequence characterizing at least a predicted binding affinity of the binder protein specified by the current amino acid sequence for the binding target;
determining an acceptance probability based on: (i) the design score for the alternative amino acid sequence, (ii) the design score for the current amino acid sequence, and (iii) a temperature parameter, wherein higher values of the temperature parameter determine a higher acceptance probability even if the design score for the alternative amino acid sequence is inferior to the design score for the current amino acid sequence; and
updating the current amino acid sequence of the binder protein to the alternative amino acid sequence with probability defined by the acceptance probability; and
selecting, using the one or more computers, an output amino acid sequence for the binder protein of the drug that targets the binding target based on the design scores generated over the plurality of iterations, wherein the output amino acid sequence has a superior design score as compared to a plurality of other amino acid sequences generated over the plurality of iterations; and
providing the selected output amino acid sequence for the binder protein for synthesis.

21. The non-transitory computer storage media of claim 20, wherein determining the design score comprises:
determining a similarity between: (i) the predicted structure of the protein specified by the alternative amino acid sequence, and (ii) a goal protein structure; and
determining the design score based at least in part on the similarity between: (i) the predicted structure of the protein specified by the alternative amino acid sequence, and (ii) the goal protein structure.

22. The non-transitory computer storage media of claim 21, wherein determining the similarity between: (i) the predicted structure of the protein specified by the alternative amino acid sequence, and (ii) the goal protein structure, comprises:
determining a similarity between: (i) one or more secondary structure elements of the predicted structure specified by the alternative amino acid sequence, and (ii) one or more secondary structure elements of the goal protein structure.

23. (canceled)

24. (canceled)

25. The non-transitory computer storage media of claim 20, wherein the binding target comprises a target protein.
